(19) United States
(12) Patent Application Publication
(10) Pub. No.: US 2025/0094661 A1
(43) Pub. Date: Mar. 20, 2025

(54) MULTI-SCALE SIMULATION-BASED FUNCTIONALLY GRADED MATERIAL DESIGN DEVICE AND METHOD FOR HIGH TEMPERATURE ENVIRONMENT

(71) Applicant: FOUNDATION OF SOONGSIL UNIVERSITY-INDUSTRY COOPERATION, Seoul (KR)

(72) Inventors: Ji Woong KIM, Seoul (KR); Hyo Kyeong KIM, Seoul (KR); Myung Jae KIM, Seoul (KR); Jin Yong LEE, Seoul (KR); Soo Ah KYUNG, Seoul (KR)

(21) Appl. No.: 18/437,339

(22) Filed: Feb. 9, 2024

(30) Foreign Application Priority Data
Sep. 19, 2023 (KR) 10-2023-0124726

(51) Int. Cl. G06F 30/17; G06F 30/20

(52) U.S. Cl. CPC G06F 30/17; G06F 30/20

(57) ABSTRACT

One embodiment provides a multi-scale simulation-based functionally graded material design method for a high temperature environment. According to one embodiment, since the characteristics of a functionally graded material (FGM) may be predicted through multi-scale simulation in advance and optimal conditions may be derived, it is possible to reduce the costs required for experiment and manufacture and shorten the manufacturing time.

### CROSS REFERENCE TO RELATED APPLICATION

The present application claims priority to Korean Patent Application No. 10-2023-0124726, filed Sep. 19, 2023, the entire contents of which is incorporated herein for all purposes by this reference.

### BACKGROUND OF THE INVENTION

### Field of the Invention

The present invention relates to a multi-scale simulation-based functionally graded material design method for a high temperature environment.

### Description of the Related Art

In the case of physical vapor deposition (PVD) thin film design and development, research and development has been conventionally conducted by a method of empirically directly forming a thin film and then checking the formed thin film, which has a problem of being highly dependent on empirical knowledge and traditional methods and showing very low efficiency in terms of time and cost.

In addition, in conventional thin film design process, there is a problem that a delaminating phenomenon occurs, since there is no intermediate layer between the main layer(upper coating layer) and the substrate (base material) that reduced the difference in the thermal expansion coefficient.

In order to solve the above problems, it is possible to suppress the delaminating phenomenon through a functionally graded material method of reducing a gap of thermal expansion of between the main layer and the substrate, but in order to manufacture functionally graded materials (FGMs), there is a problem that much time and money are consumed in a process of selecting and experimenting a composition.

Therefore, many challenges for the development for a high-efficiency thin film design technology using a computational design-based FGM design method still remain.

### DOCUMENTS OF RELATED ART

(Patent Document 1) Korean Patent Application Laid-Open No. 10-2020-0086746

### SUMMARY OF THE INVENTION

The present invention is directed to providing a method of designing a functionally graded material (FGM) in a high temperature environment using multi-scale simulation.

The objects of the present invention are not limited to the above-described objects, and other objects that are not mentioned will be able to be clearly understood by those skilled in the art to which the present invention pertains from the following description.

In order to achieve the object, one embodiment of the present invention provides a multi-scale simulation-based functionally graded material design device for a high temperature environment.

A multi-scale simulation-based functionally graded material design device for a high temperature environment according to one embodiment of the present invention includes an input unit configured to input information about each of a base material and an upper coating layer, a functionally graded material composition design unit configured to select a candidate group of the upper coating layer input to the input unit, determine a structure of a functionally graded material through structural optimization of a functionally graded material composition positioned between the base material and the upper coating layer, and calculate a physical property value, a modeling unit configured to analyze a thermal stress according to composition and thickness arrangement of the functionally graded material by performing multi-scale simulation based on the physical property value calculated by the functionally graded material composition design unit, and an output unit configured to output a composition and thickness of a candidate group of the functionally graded material based on a result of the modeling.

In addition, according to one embodiment of the present invention, the information input from the input unit may be composition information.

In addition, according to one embodiment of the present invention, in the input unit, a method of selecting the candidate group of the upper coating layer may include calculating formation energy according to a content when a transition metal is doped in a specific structure of the input information, then finding a stable phase, and calculating a physical property value.

In addition, according to one embodiment of the present invention, in the functionally graded material composition design unit, a method of optimizing a structure of the functionally graded material composition may include determining a structure with the lowest energy by moving atoms of molecules through the structural optimization using a density functional theory (DFT) with respect to a composition and structure thereof, thereby obtaining information about phase stability.

In addition, according to one embodiment of the present invention, in the functionally graded material composition design unit, the physical property value may include a Young's modulus, a Poisson's ratio, and a density value derived through DFT calculation.

In addition, according to one embodiment of the present invention, in the modeling unit, finite element analysis (FEA) may be performed based on the physical property value obtained from the functionally graded material composition design unit.

In order to achieve the object, another embodiment of the present invention provides a multi-scale simulation-based functionally graded material design method for a high temperature environment.

A multi-scale simulation-based functionally graded material design method for a high temperature environment according to another embodiment of the present invention includes an operation of inputting information about each of a base material and an upper coating layer and selecting a candidate group of the upper coating layer, a functionally graded material composition designing operation of determining a structure of a functionally graded material through structural optimization of a functionally graded material composition positioned between the base material and the upper coating layer and calculating a physical property value, a modeling operation of analyzing a thermal stress according to composition and thickness arrangement of the functionally graded material by performing multi-scale simulation based on the physical property values calculated in the functionally graded material composition designing operation, and an outputting operation of outputting a composition and thickness of a candidate group of the functionally graded material based on a result of the modeling.

In addition, according to one embodiment of the present invention, the base material and the upper coating layer may have different coefficients of thermal expansion.

In addition, according to one embodiment of the present invention, the information input in the input operation may be composition information.

In addition, according to one embodiment of the present invention, in the functionally graded material composition designing operation, a method of selecting the candidate group of the upper coating layer may calculate formation energy according to a content when a transition metal is doped in a specific structure of the input information, then find a stable phase, and calculate a physical property value.

In addition, according to one embodiment of the present invention, in the functionally graded material composition designing operation, a method of optimizing a structure of the functionally graded material composition determines a structure with the lowest energy by moving atoms of molecules through the structural optimization using a density functional theory (DFT) with respect to a composition and structure thereof.

In addition, according to one embodiment of the present invention, in the functionally graded material composition designing operation, a cluster expansion technique, a special quasi-random structure (SQS) technique, a virtual crystal approximation (VCA) technique, or the like may be used.

In addition, according to one embodiment of the present invention, in the functionally graded material composition designing operation, the physical property value may include a Young's modulus, a Poisson's ratio, and a density value derived through DFT calculation.

In addition, according to one embodiment of the present invention, in the modeling operation, finite element analysis (FEA) may be performed based on the physical property value obtained from the functionally graded material composition designing operation.

### BRIEF DESCRIPTION OF THE DRAWINGS

FIG. 1 is a flowchart illustrating a multi-scale simulation-based functionally graded material design method for a high temperature environment.

FIG. 2 includes (a) a schematic diagram illustrating a conventional thin film design process, and (b) a schematic diagram illustrating the multi-scale simulation-based functionally graded material design method for a high temperature environment according to the present invention.

FIG. 3 is a schematic diagram illustrating DFT calculation.

FIG. 4 is an exemplary view illustrating a finite element method.

FIG. 5 is a graph illustrating a coefficient of thermal expansion for each temperature.

FIG. 6 is a schematic diagram illustrating a process of performing modeling by performing multi-scale simulation.

FIG. 7 is a schematic diagram illustrating selecting desired composition and physical property information from a database.

FIG. 8 is a schematic diagram illustrating a specific embodiment in which a functionally graded material is manufactured through the multi-scale simulation-based functionally graded material design method for a high temperature environment.

### DETAILED DESCRIPTION OF THE INVENTION

Hereinafter, the present invention will be described with reference to the accompanying drawings. However, the present invention may be implemented in various different forms and is not limited to embodiments described herein. In addition, in order to clearly describe the present invention in the drawings, components irrelevant to the description have been omitted, and throughout the specification, similar components have been denoted by similar reference numerals.

Throughout the specification, when a first component is described as being “connected to (joined to, in contact with, or coupled to)” a second component, this includes not only a case in which the first component is “directly connected” to the second component, but also a case in which the first component is “indirectly connected” to the second component with a third component interposed therebetween. In addition, when the first component is described as “including,” the second component, this means that the first component may further include the third component rather than precluding the third component unless especially stated otherwise.

The terms used in the specification are only used to describe specific embodiments and are not intended to limit the present invention. The singular expression includes the plural expression unless the context clearly dictates otherwise. In the specification, it should be understood that terms such as “comprise” or “have” are intended to specify that a feature, a number, a step, an operation, a component, a part, or a combination thereof described in the specification is present, but do not preclude the possibility of the presence or addition of one or more other features, numbers, steps, operations, components, parts, or combinations thereof.

Hereinafter, the present invention will be described with reference to the accompanying drawings suggested in the specification. For reference, the drawings may be somewhat exaggerated to describe the features of the present invention. In this case, it is preferable that the drawings should be construed in light of all purposes of the specification.

In the case of the design and development for a conventional physical vapor deposition (PVD) thin film, since there is a problem that a delaminating phenomenon occurs due to a difference in thermal expansion due to the absence of an intermediate layer for reducing a space between a main layer and a substrate, it is possible to suppress the delaminating phenomenon by reducing the difference in thermal expansion through a functionally graded material method of reducing a gap between the main layer and the substrate, but in order to manufacture the functionally graded material, there is a problem that much time and money are consumed in a process of selecting and experimenting a composition. In order to solve the above problems, the present invention provides a multi-scale simulation-based functionally graded material (FGM) design method for a high temperature environment.

A multi-scale simulation-based functionally graded material design device for a high temperature environment according to one embodiment of the present invention will be described.

The multi-scale simulation-based functionally graded material design device for a high temperature environment according to one embodiment of the present invention includes an input unit for inputting information about each of a base material and an upper coating layer, a functionally graded material composition design unit for selecting a candidate group of the upper coating layer input to the input unit, determining a structure of a functionally graded material through structural optimization of a functionally graded material composition positioned between the base material and the upper coating layer, and calculating a physical property value, a modeling unit for analyzing a thermal stress according to composition and thickness arrangement of the functionally graded material by performing multi-scale simulation based on the physical property value calculated by the functionally graded material composition design unit, and an output unit for outputting a composition and thickness of a candidate group of the functionally graded material based on a result of the modeling.

The present invention may include the input unit.

In this case, the input unit according to the present invention inputs the information about each of the base material and the upper coating layer.

In this case, the information input from the input unit is composition information.

In this case, the base material and the upper coating layer in the input unit may have different coefficients of thermal expansion.

In addition, the present invention may include a composition design unit.

In this case, the candidate group for the upper coating layer input to the input unit may be selected, the structure of the functionally graded material composition may be determined through the structural optimization of the functionally graded material composition positioned between the base material and the upper coating layer, and the physical property value may be calculated.

In this case, the method of selecting the candidate group of the upper coating layer may include calculating formation energy according to a content when a transition metal is doped in a specific structure of the input information, then finding a stable phase, and calculating a physical property value.

In addition, the method of optimizing the structure of the functionally graded material composition may include determining a structure with the lowest energy by moving atoms of molecules through the structural optimization using a density functional theory (DFT).

In addition, as the physical property value, a Young's modulus, a Poisson's ratio, and a density value may be derived through DFT calculation.

In addition, the present invention may include the modeling unit.

In this case, the modeling unit may be connected to the composition design unit and may analyze a thermal stress according to the composition and thickness arrangement of the functionally graded material by performing multi-scale simulation based on the physical property value of the functionally graded material.

In this case, the compositions and thicknesses of the candidate group of the functionally graded material may be output based on a result of the modeling.

In addition, finite element analysis (FEA) may be performed based on the physical property value obtained from the functionally graded material composition design unit.

In addition, the present invention may include the output unit.

In this case, the output unit may be connected to the modeling unit and may output the compositions and thicknesses of the candidate group of the functionally graded material based on a result of the modeling.

In this case, the multi-scale simulation-based functionally graded material design method for a high temperature environment will be described below in detail in the multi-scale simulation-based functionally graded material design method for a high temperature environment.

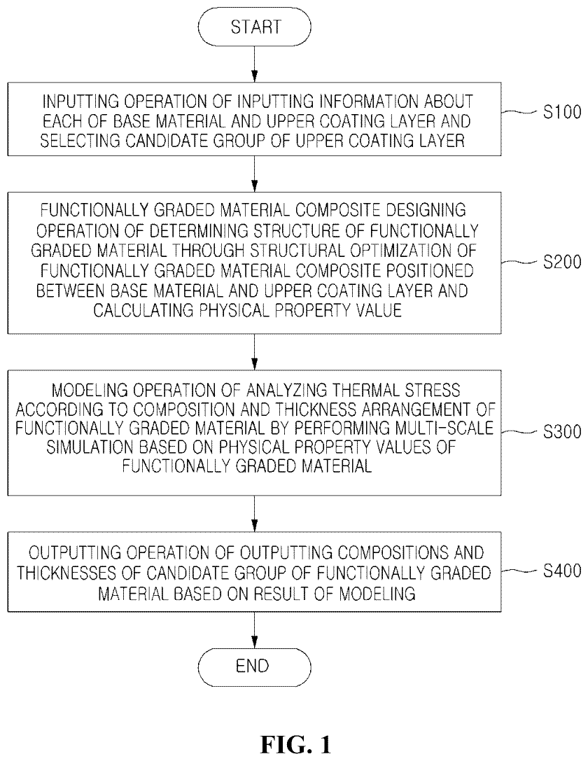

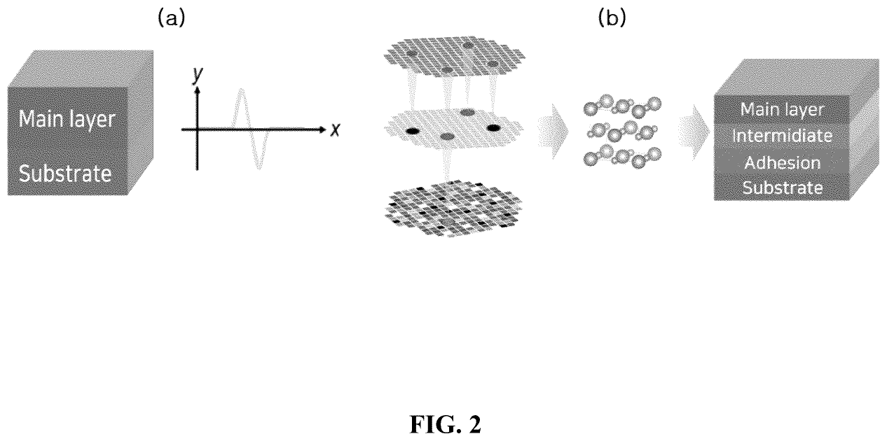

The multi-scale simulation-based functionally graded material design method for a high temperature environment according to one embodiment of the present invention will be described with reference to FIGS. 1 and 2.

FIG. 1 is a flowchart illustrating a multi-scale simulation-based functionally graded material design method for a high temperature environment.

FIG. 2 includes (a) a schematic diagram illustrating a conventional thin film design process, and (b) a schematic diagram illustrating the multi-scale simulation-based functionally graded material design method for a high temperature environment according to the present invention.

The multi-scale simulation-based functionally graded material design method for a high temperature environment according to one embodiment of the present invention uses the above-described multi-scale simulation-based functionally graded material design device for a high temperature environment and includes,
an inputting operation of inputting information about each of the base material and the upper coating layer (S100), a functionally graded material composition designing operation of determining the structure of the functionally graded material through the structural optimization of the functionally graded material composition positioned between the base material and the upper coating layer and calculating the physical property value (S200), a modeling operation of analyzing the thermal stress according to the composition and thickness arrangement of the functionally graded material by performing the multi-scale simulation based on the physical property values of the functionally graded material (S300), and an outputting operation of outputting the compositions and thicknesses of the candidate group of the functionally graded material based on a result of the modeling (S400).

A first operation may include the inputting operation of inputting the information about each of the base material and the upper coating layer and selecting the candidate group of the upper coating layer (S100).

The present invention is directed to solving the conventional problem that the delaminating phenomenon occurs due to the difference in thermal expansion between the base material and the upper coating layer, and the base material and the upper coating layer have different coefficients of thermal expansion.

In this case, the information input in the inputting operation is composition information.

In this case, the base material input in the inputting operation uses WC-Co or a cermet (ceramic-metal composite) mixed with Ti(C,N) and Ni, but is not limited to the above-described examples.

In addition, a content of the mixture may be adjusted in the inputting operation.

For example, in WC-Co, a percentage of Co to be added may be determined.

In addition, in TiC or Ti(C,N) and Ni-based cermet (ceramic-metal composite), a percentage of Ni to be added may be fdetermined.

In this case, the cermet is a heat-resistant material made of a metal and ceramic made by a powder metallurgy. The cermet is sintered in hydrogen, vacuum, or other suitable atmosphere and is a new material that has both the characteristics of ceramics such as hardness, heat resistance, oxidation resistance, chemical resistance, and wear resistance and the toughness, plasticity, and mechanical strength of a metal.

In addition, the upper coating layer input in the inputting operation uses one or more materials selected from the group consisting of (Ti₁₋ₓAlₓ)N, TiN, CrN, and AlN, and an x value may be determined in the inputting operation.

In the operation of selecting the candidate group of the upper coating layer, the candidate group may be selected and extracted based on the input information. In the second operation, a database on the base material and a main coating layer used may be used.

In this case, for example, when the upper coating layer is made of (Ti₁₋ₓAlₓ)N, in the case of the input (Ti₁₋ₓAlₓ)N main coating layer, the higher a content of Ti, the more dominant a cubic structure may be, and the higher a content of Al, the more dominant a hexagonal structure may be.

In this case, the formation energy according to the content of Al may be calculated, the stable phase may be found, and data on structural information and mechanical physical properties of the corresponding structure may be produced.

In this case, the operation of selecting the candidate group of the upper coating layer may include a structure optimizing operation of calculating the formation energy according to the content when the transition metal is doped in the specific structure of the material input in the inputting operation and finding the stable phase, and an operation of selecting an optimal candidate group by calculating the physical property of the optimal structure derived in the structure optimization operation.

Specifically, in the operation of selecting the optimal candidate group of the upper coating layer, used physical property values may vary depending on the purpose of the upper coating layer.

For example, when the upper coating layer should be used in an application where oxidation resistance is important, an oxidation of the base material should be calculated, and when an upper coating layer (main layer) for a high temperature environment should be developed, the deterioration of the physical property at high temperatures should be minimal.

In addition, when there is a need for the development for a high hardness thin film, the hardness should be high.

As described above, the calculated physical properties are different depending on each application.

In this case, for example, in the case of developing a thin film for a cutting tool capable of cutting at high speed, hardness is most important in the upper coating layer.

In this case, specific calculation equations for selecting the optimal candidate group of the upper coating layer are as expressed in Equations 2 to 5 below. In Equation 1, a shear modulus of elasticity and a bulk modulus of elasticity were used, and method for calculating each are expressed in Equations 2 and 5. V, R, and H denote a Voigt, Reuss, and Hill moduli of elasticity, respectively.

$${H}_{v} = 2 {\{ {( {G}_{H} / {B}_{H} )}^{2} G \}}^{0.585} - 3 \qquad [ Equation 1 ]$$

$${G}_{H} = \frac{{G}_{V} + {G}_{R}}{2} \qquad [ Equation 2 ]$$

$${G}_{V} = \frac{3 {C}_{44} + {C}_{11} - {C}_{12}}{5} \qquad [ Equation 3 ]$$

$${G}_{R} = \frac{5 ( {C}_{11} - {C}_{12} ) {C}_{44}}{4 {C}_{44} + 3 ( {C}_{11} - {C}_{12} )} \qquad [ Equation 4 ]$$

$${B}_{H} = \frac{{C}_{11} + 2 {C}_{12}}{3} \qquad [ Equation 5 ]$$

In this case, C₁₁ and C₁₂ denote indices representing a change in volume in response to a force applied perpendicularly to a surface of a crystal structure, and C₁₄ denotes a degree of change when the shear deformation of a material occurs. Therefore, C₁₁ and C₁₂ are used to calculate the bulk modulus of elasticity representing resistance to compression, and C₄₄₋ together with C₁₁ and C₁₂₋ are used to calculate the shear modulus of elasticity and a Young's modulus, which represent resistance to the shear deformation.

A second operation may include the functionally graded material composition designing operation of determining the structure of the functionally graded material through the structural optimization of the functionally graded material composition positioned between the base material and the upper coating layer and calculating the physical property (S200).

The functionally graded material (FGM) according to the present invention is a material in which a component and a composition are consecutively changed in a thickness direction of the material and is a special heterogeneous composite material that can implement special functions due to a difference between contents of various heterogeneous materials. In other words, the functionally graded material is a material in which a mixing ratio of two or more materials gradually changes depending on positions to form a gradient.

Conventionally, research and development has been conducted in an empirical manner to select the above-described functionally graded material, and there is a problem that much time and money are consumed in the process of selecting and experimenting the composition to produce the functionally graded material.

Therefore, in order to solve the above problem, according to the present invention, the functionally graded material may be designed based on a computational design, and a candidate group of an element that may be used as the functionally graded material may be selected based on a coefficient of thermal expansion, a difference between sizes of lattices, an elastic constant, or the like.

In this case, the candidate group of the element that may be used as the functionally graded material may be selected based on the coefficient of thermal expansion of the functionally graded material composition.

In this case, specifically, the process of selecting the candidate group of the element that may be used as the functionally graded material is referred to as a FGM design operation and a physical property calculating operation and specifically includes an operation of calculating the coefficient of thermal expansion between materials, an operation of calculating the formation energy and confirming phase stability, and selecting an element by first performing calculating the structural and mechanical properties of the selected composition.

Therefore, when the candidate group of the element that may be used as the functionally graded material is selected, the element that may be used as the functionally graded material may be found without an actual experiment.

For example, an element that may be added to the functionally graded material may be Ti, Hf, Zr, Nb, or Al.

In this case, in the functionally graded material composition designing operation, a cluster expansion technique, a special quasi-random structure (SQS) technique, and a virtual crystal approximation (VCA) technique may be used.

The cluster expansion technique is widely used to study materials that show substitution disorder where some crystal sites may be occupied by one or more types of atoms. The cluster expansion technique is replaced with an individual “site” variable that represents which atoms (or vacancies) are present at a particular site. The cluster expansion technique is a technique that helps construct the structure-property relationship of the material and accelerate the discovery of new materials through a rational design.

In addition, the special quasi-random structure (SQS) technique generates a supercell with a minimum size close to a random (disordered) solid solution. The following operations are performed. There are an operation of generating an appropriate input file, an operation of calculating a target correlation of a random structure, an operation of generating a structure with a specific size based on the target correlation, and finally, an operation of evaluating a candidate structure based on a correlation with the number of increased clusters.

In this case, in the composition design operation, the composition and structure of the functionally graded material may be designed by performing a method of determining a structure of the composition using the most stable structure having minimum energy by moving the atoms of the molecules through the structural optimization using the DFT from the candidate group of the element.

Specifically, the method of optimizing the structure using the DFT may include performing calculation under the following condition, and the composition and structure of the functionally graded material composition may be designed by performing the calculation.

DFT-based first-principles calculation was performed based on vienna ab initio simulation package (VASP) code. In order to consider a change in electron distribution probability according to distance between electrons and an atomic nucleus, the generalized gradient approximation with the Perdew-Burke-Ernzerhof (GGA-PBE) exchange correlation functional was used as an exchange correlation energy equation.

In this case, a cutoff energy value of 500 eV was used to consider a plane wave at high energy levels, and the optimization of all structures was performed when an inter-atomic force was 0.015 eV/A or less and at the same time, electron energy was stabilized to 10 eV or less. Brillouin zone integration used Monkhorst-Pack often used in the DFT calculation.

In the physical property calculating operation according to the present invention, a Young's modulus, a Poisson's ratio, and a density value may be derived through the DFT calculation.

An elastic constant may be calculated to derive the Young's modulus, the Poisson's ratio, and the density value.

The elastic constant is a value that varies depending on a crystal structure, and a physical property may be calculated by deriving the Young's modulus, the Poisson's ratio, and the density value through the elastic constant.

First, the elastic constant may be derived through strain-stress relation calculation, and detailed calculation equations are as expressed in Equations 6 and 7 below.

$${σ}_{ij} = {C}_{ijkl} {ε}_{kl} \qquad [ Equation 6 ]$$

$$( \begin{matrix}{σ}_{1} \\ {σ}_{2} \\ {σ}_{3} \\ {τ}_{4} \\ {τ}_{5} \\ {τ}_{6}\end{matrix} ) = ( \begin{matrix}{C}_{11} & {C}_{12} & {C}_{12} & 0 & 0 & 0 \\ {C}_{12} & {C}_{11} & {C}_{12} & 0 & 0 & 0 \\ {C}_{12} & {C}_{12} & {C}_{11} & 0 & 0 & 0 \\ 0 & 0 & 0 & {C}_{44} & 0 & 0 \\ 0 & 0 & 0 & 0 & {C}_{44} & 0 \\ 0 & 0 & 0 & 0 & 0 & {C}_{44}\end{matrix} ) ( \begin{matrix}{ε}_{1} \\ {ε}_{2} \\ {ε}_{3} \\ {γ}_{4} \\ {γ}_{5} \\ {γ}_{6}\end{matrix} ) \qquad [ Equation 7 ]$$

In Equation 6, σᵢⱼ denotes a stress tensor, Cᵢⱼₖₗ denotes a Lagrangian strain tensor, and εₖₗ denotes an elastic constant tensor that is a matrix. In the case of a cubic structure, C₁₁, C₁₂, and C₄₄ were mainly confirmed. In this case, Cᵢⱼ denotes the elastic constant, and σᵢ and τⱼ denote a normal stress and a shear stress, respectively.

In addition, according to the present invention, the Young's modulus, the Poisson's ratio, and the density value may be derived through the first-principles calculation, and a detailed calculation equation of the Young's modulus derived through the first-principles calculations is as expressed in Equation 8 below.

$${E}_{H} = \frac{9 {G}_{H} {B}_{H}}{3 {B}_{H} + {G}_{H}} \qquad [ Expression 8 ]$$

In addition, a detailed calculation equation of the Poisson's ratio derived through the first-principles calculation is as expressed in Equation 9 below.

$$v = ( 3 {B}_{H} - 2 {G}_{H} ) / 2 ( 3 {B}_{H} + {G}_{H} ) \qquad [ Expression 9 ]$$

In addition, a detailed calculation equation of the density derived through the first-principles calculation is as expressed in Equation 10 below.

$$Density = Mass / Volume \qquad [ Expression 10 ]$$

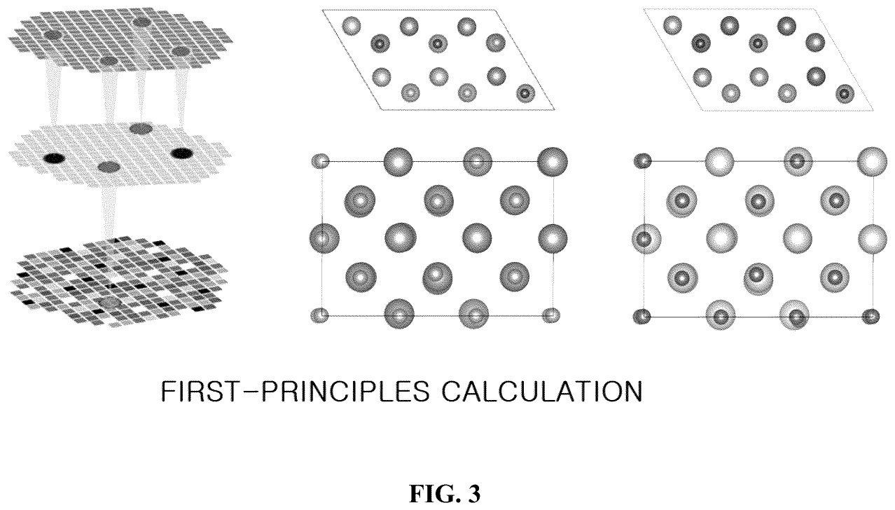

In this case, FIG. 3 is a schematic diagram illustrating first-principles calculation. Referring to FIG. 3, information about the Young's modulus, Poisson's ratio, and density of the functionally graded material composition may be confirmed through the first-principles calculation.

The Young's modulus, Poisson's ratio, and density value derived through the above calculation may represent structural and mechanical physical property values.

In this case, the Young's modulus is a coefficient that represents how a relative length of an elastic object is changed by a stress, is referred to as an elastic modulus, and is a physical property value necessary to check how well the functionally graded material is bent.

In addition, the Poisson's ratio is a ratio of a horizontal deformation and vertical deformation caused by a vertical stress generated inside a material and is a constant value for the same material within an elastic limit. This value is constant only when the material is homogeneous, isotropic, or orthotropic, and an acting axial force is constant in all sections of a length of the material.

In this case, the reason the Poisson's ratio value is calculated in the present invention is because the Poisson's ratio value is a value input as input unit in the FEA.

In addition, the density is a value that represents mass per unit volume, and when a volume is constant, the greater a density of an object, the greater a mass of the object. An average density of the object is equal to a value obtained by dividing the total mass by the total volume, and a denser object has a smaller volume than a less dense object with the same mass. The density value can represent how much mass of material is contained in a constant area in the material.

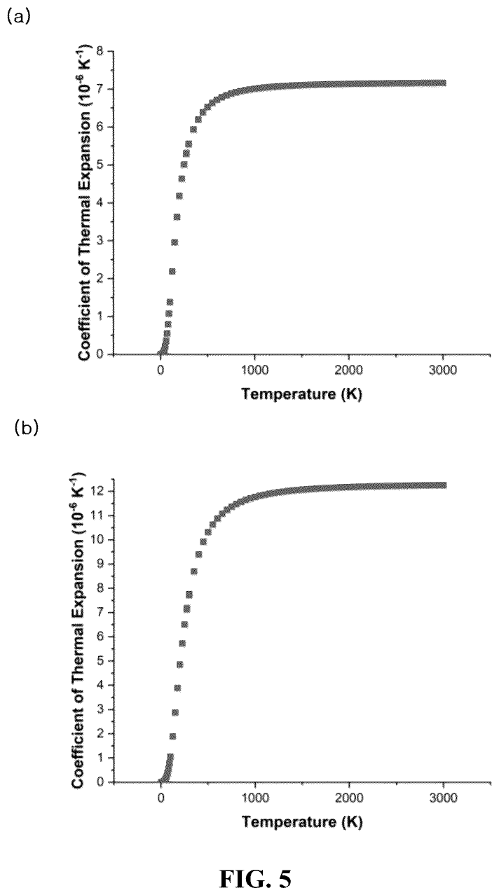

In this case, FIG. 5 is a graph illustrating a coefficient of thermal expansion for each temperature.

Referring to FIG. 5, a coefficient of thermal expansion for each temperature can be seen, and through FIG. 5, it can be seen that when a temperature of a material changes, a volume changes, and a solid such as a metal is expressed with a length change rate rather than a volume change rate, a coefficient of thermal expansion is a value representing the length change rate, a change in length is proportional to the total length and a change in temperature, and as the temperature increases and the length increases, the change in length increases.

Therefore, the structural and mechanical physical properties may be calculated through the first-principles calculation.

A third operation may include the modeling operation of analyzing the thermal stress according to the composition and thickness arrangement of the functionally graded material by performing the multi-scale simulation based on the physical property values of the functionally graded material (S300).

In the modeling operation, the FEA may be performed based on the physical property values obtained in the functionally graded material composition designing operation.

The FEA is a computer simulation technique and a computing method of predicting how a product reacts to an actual force, vibration, heat, a fluid flow, and other physical effects. The FEA shows whether a product is broken and worn under a preset condition or is performed as designed.

The FEA is used to predict what will happen when a product is used. In addition, the FEA is performed by a method of meshing an actual object into a large number (thousands to hundreds of thousands) of finite elements, such as small cubes.

In this case, an operation of each element is predicted through a mathematical formula, and a computer sums operations of all individual elements to predict the operation of the actual object.

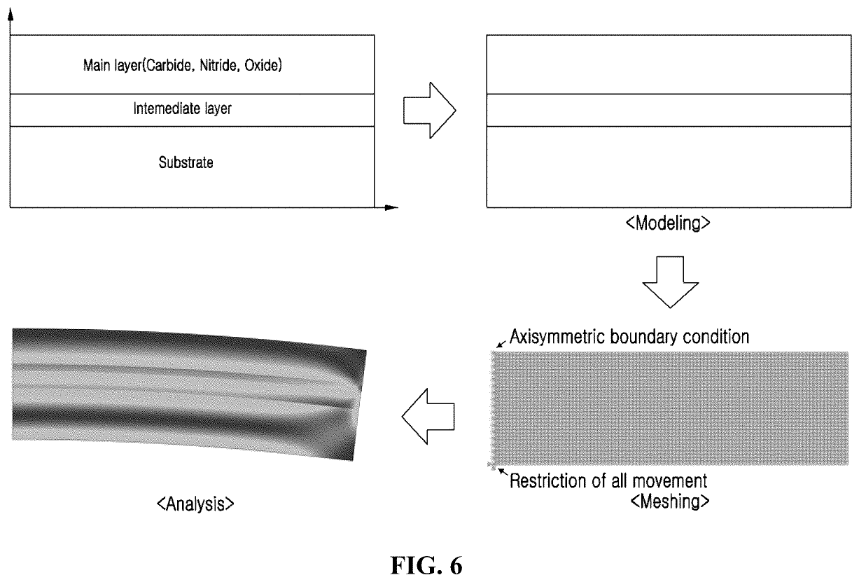

In the present invention, FIG. 6 is a schematic diagram illustrating a process of performing modeling by performing multi-scale simulation.

Referring to FIG. 6, it can be seen that a model has been meshed into thousands to hundreds of thousands of elements through the FEA. In this case, the computer predicts an operation of an object by aggregating individual operations of all components. A specific modeling process is as follows.

First, modeling was performed by using a 3D program to implement the simulation. Then, physical property data suitable for each section of the model were input.

Next, the model was meshed into thousands to hundreds of thousands of elements.

In this case, the mesh is an element network of the model required when performing structural analysis in the FEA and represents an approximation of an actual shape of a structure.

In addition, the present invention is characterized by performing the modeling through the FEA, and a load or displacement may be applied to the model by boundary conditions.

Next, the boundary conditions were specified for each axis, and a temperature and load to be simulated were assigned.

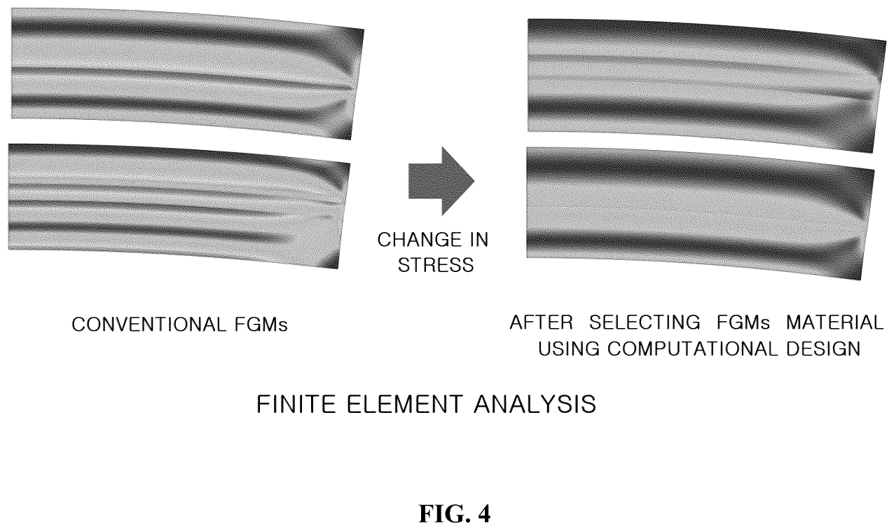

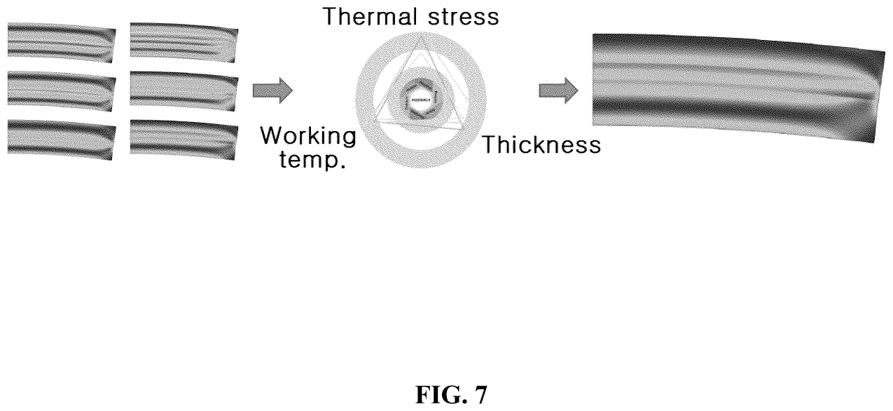

In addition, referring to FIGS. 4 and 7, it can be seen that a material physical property selection system may be constructed by sequentially automating the entire calculation process from model generation to final selection.

Therefore, according to the present invention, it is possible to reduce the development period and cost by selecting the composition and physical properties of materials with desired conditions in a generated database and suggest materials for a desired optimized thin film.

In other words, it can be seen that the optimized thin film is a visually optimized thin film through an FEA system.

A fourth operation may include the outputting operation of outputting the compositions and thicknesses of the candidate group of the functionally graded material based on a result of the modeling (S400).

In the outputting operation, the composition and thickness information about the candidate group derived based on a result of the modeling is output.

In this case, information, such as a thermal stress, a maximum stress position, and a change amount, according to a temperature and thickness of a layer on which the functionally graded material is formed can be seen through the output information.

Therefore, since the multi-scale simulation-based functionally graded material design method for a high temperature environment according to the present invention can predict the characteristics of the functionally graded material (FGM) through the multi-scale simulation in advance and derive the optimal conditions, it is possible to reduce the costs required for experiment and manufacture and shorten the manufacturing time.

In addition, when the functionally graded material (FGM) is designed, it is possible to minimize the time and cost consumed in the process of selecting the composition.

In addition, when the functionally graded material (FGM) is derived through the multi-scale simulation, it is possible to prevent defects and improve the stability and functionality of the functionally graded material (FGM).

Hereinafter, the present invention will be described in more detail through a manufacturing example and an experimental example. The manufacturing example and experimental example are merely for exemplarily describing the present invention, and the scope of the present invention is not limited by the manufacturing example and experimental example.

### Example: The Multi-Scale Simulation-Based Functionally Graded Material Design Method for a High Temperature Environment

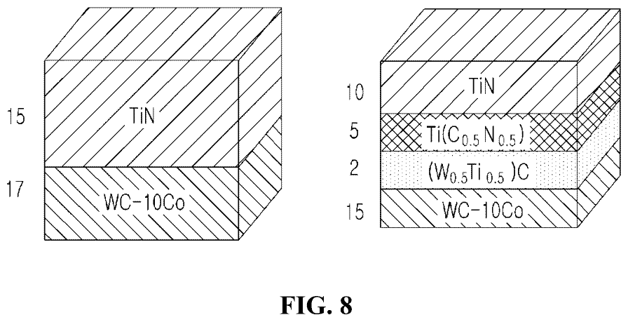

The multi-scale simulation-based functionally graded material design method for a high temperature environment according to one embodiment of the present invention will be described with reference to FIG. 8.

First, composition information about the base material made of a WC-10Co material and composition information about the upper coating layer made of a TiN material were input to the database.

Next, Ti(C_{0.5}N_{0.5}) and (Ti_{0.5}W_{0.5})C, which were the optimal candidate group (functionally graded materials) of the upper coating layer, were selected by performing the method of calculating the formation energy according to the content when the transition metal was doped in the specific structure of the input information, then finding the stable phase, and calculating the physical property value.

Next, the structure of the functionally graded material composition was searched by determining the structure of the composition with the most stable structure with the lowest energy by moving the atoms of the molecules through structure optimization using the DFT.

In this case, the determined structure of the functionally graded material composition is a face centered cubic lattice (FCC) structure.

Next, internal energy of the functionally graded material composition searched in the composition design operation was calculated, information about phase stability was obtained, and then physical property values such as a Young's modulus, a Poisson's ratio, a density, and a coefficient of thermal expansion were derived. Specifically, the formation energy according to the content when the transition metal is doped in the specific structure of the input information may be calculated, then the stable phase may be found, and the physical property value may be calculated.

Next, the behavior of the object was predicted and modeled by performing the multi-scale simulation on the material under a periodic boundary condition (PBC) through the FEA based on the physical property values.

The substrate was fixed to 15 μm and a width thereof was 100 μm. Each coating layer was designed like a multi-layer structure. The FGM coating layer also has the same width, but each layer is designed to have a different length and has a length of 2 to 5 μm. This model used a CPS4R element and included about 3333 nodes, Global Seeds was set to 1 μm, and a high density mesh was used. The left part of the model was used as anoriginal axis and restricted on an x-axis (x=0, U1=0), and a vertical displacement was restricted at a bottom left coordinate origin of the model (x=0, y=0, U1=U2=UR3=0). All other edges were set to be bent when the temperature changed, and a thermal stress in a layer/substrate system was generated by applying a uniform temperature load to the model.

Next, the information about the physical properties of the composition according to temperatures was output, and a functionally graded material was formed between the base material and the upper coating layer.

### Manufacturing Example: Substrate Manufactured Through the Multi-Scale Simulation-Based Functionally Graded Material Design Method for a High Temperature Environment

First, the base material made of a WC-10Co material was formed to a thickness of 15 m through the “Model” and “Part” modules among the FEA programs.

Next, the TiN thin film layer (upper coating layer) was formed above the base materialin a thickness of 17 μm through the “Model” and “Part” modules among the FEA programs.

Next, a first functionally graded material with a thickness of 2 μm and a second functionally graded material with a thickness of 5 μm were formed between the base material and the upper coating layer through the “Model” and “Part” modules among the FEA programs.

### Experimental Example 1: Experiment for Checking the Multi-Scale Simulation-Based Functionally Graded Material Design Method for a High Temperature Environment

The present invention was experimented under two conditions: 1,000 K and 1,500 K.

The maximum stress of the conventional thin film was 905 MPa, while the maximum stress of the functionally graded material was 738 MPa, resulting in a difference therebetween.

For example, referring to FIG. 6, it can be seen that a stress applied to an interface or the like is excellent even at a high temperature of 1,500 K.

As a result of the FEA, it can be seen that the multi-layer structure having the functionally graded material, which is the feature of the present invention, significantly affects the improvement in the overall structural, thermal, and mechanical characteristics through the excellent thermal characteristics at the interface.

Since the multi-scale simulation-based functionally graded material design method according to one embodiment of the present invention can predict the characteristics of the functionally graded material (FGM) through the multi-scale simulation in advance and derive the optimal conditions, it is possible to reduce the costs required for experiment and manufacture and shorten the manufacturing time.

In addition, when the functionally graded material (FGM) is designed, it is possible to minimize the time and cost consumed in the process of selecting the composition.

In addition, when the functionally graded material (FGM) is derived through the multi-scale simulation, it is possible to prevent defects and improve the stability and functionality of the functionally graded material (FGM).

It should be understood that the effects of the present invention are not limited to the above-described effects and include all effects inferrable from the configuration of the invention described in the detailed description or claims of the present invention.

The above description of the present invention is for illustrative purpose, and those skilled in the art to which the present invention pertains will be able to understand that the present invention may be easily modified in other specific forms without changing the technical spirit or essential features thereof. Therefore, it should be understood that the above-described embodiments are illustrative and not restrictive in all respects. For example, each component described in a singular form may be implemented separately, and likewise, components described as being implemented separately may also be implemented in a combined form.

The scope of the present invention is defined by the claims to be described below, and all changes or modifications derived from the meaning and scope of the claims and equivalent concepts thereof should be construed as being included in the scope of the present invention.

1. A multi-scale simulation-based functionally graded material design device for a high temperature environment, comprising:
an input unit configured to input information about each of a base material and an upper coating layer;
a functionally graded material composition design unit configured to select a candidate group of the upper coating layer input to the input unit, determine a structure of a functionally graded material through structural optimization of a functionally graded material composition positioned between the base material and the upper coating layer, and calculate a physical property value;
a modeling unit configured to analyze a thermal stress according to composition and thickness arrangement of the functionally graded material by performing multi-scale simulation based on the physical property value calculated by the functionally graded material composition design unit; and
an output unit configured to output a composition and thickness of a candidate group of the functionally graded material based on a result of modeling of the modeling unit.

2. The multi-scale simulation-based functionally graded material design device according to claim 1, wherein the information input from the input unit is composition information.

3. The multi-scale simulation-based functionally graded material design device according to claim 1, wherein in the input unit, of selecting the candidate group of the upper coating layer includes calculating formation energy according to a content when a transition metal is doped in a specific structure of the input information, then finds-finding a stable phase, and calculating the physical property value.

4. The multi-scale simulation-based functionally graded material design device according to claim 1, wherein in the functionally graded material composition design unit, optimizing a structure of the functionally graded material composition includes determining a structure with a lowest energy by moving atoms of molecules through the structural optimization using a density functional theory (DFT) with respect to a composition and structure thereof.

5. The multi-scale simulation-based functionally graded material design device according to claim 1, wherein in the functionally graded material composition design unit, the physical property value includes a Young's modulus, a Poisson's ratio, and a density value derived through first-principles calculation.

6. The multi-scale simulation-based functionally graded material design device according to claim 1, wherein in the modeling unit, finite element analysis (FEA) is performed based on the physical property value obtained from the functionally graded material composition design unit.

7. A multi-scale simulation-based functionally graded material design method for a high temperature environment using the multi-scale simulation-based functionally graded material design device for a high temperature environment according to claim 1, the method comprising:
an inputting operation of inputting information about each of a base material and an upper coating layer;
a functionally graded material composition designing operation of selecting a candidate group of the upper coating layer input in the inputting operation, determining a structure of a functionally graded material through structural optimization of a functionally graded material composition positioned between the base material and the upper coating layer, and calculating a physical property value;
a modeling operation of analyzing a thermal stress according to composition and thickness arrangement of the functionally graded material by performing multi-scale simulation based on the physical property values calculated in the functionally graded material composition designing operation; and
an outputting operation of outputting a composition and thickness of a candidate group of the functionally graded material based on a result of the modeling operation.

8. The method according to claim 7, wherein the base material and the upper coating layer have different coefficients of thermal expansion.

9. The method according to claim 7, wherein the information input in the inputting operation is composition information.

10. The method according to claim 7, wherein in the functionally graded material composition designing operation, selecting the candidate group of the upper coating layer includes calculating formation energy according to a content when a transition metal is doped in a specific structure of the input information, then finding a stable phase, and calculating the physical property value.

11. The method according to claim 7, wherein in the functionally graded material composition designing operation, optimizing a structure of the functionally graded material composition includes determining a structure with a lowest energy by moving atoms of molecules through the structural optimization using a density functional theory (DFT) with respect to a composition and structure thereof, thereby obtaining information about phase stability.

12. The method according to claim 7, wherein in the functionally graded material composition designing operation, a cluster expansion technique or a special quasi-random structure (SQS) technique is used.

13. The method according to claim 7, wherein in the functionally graded material composition designing operation, the physical property value includes a Young's modulus, a Poisson's ratio, and a density value derived through first-principles calculation.

14. The method according to claim 7, wherein in the modeling operation, finite element analysis (FEA) is performed based on the physical property value obtained from the functionally graded material composition designing operation.
